(19) United States
(12) Patent Application Publication
(10) Pub. No.: US 2025/0032731 A1
(43) Pub. Date: Jan. 30, 2025

(54) DEVICE AND SYSTEM FOR PAIN MANAGEMENT

(71) Applicant: NEEDLECALM PTY LTD., Carnmeray (AU)

(72) Inventor: Lauren BARBER, Carnmeray (AU)

(21) Appl. No.: 18/919,361

(22) Filed: Oct. 17, 2024

Related U.S. Application Data
(63) Continuation of application No. 17/041,119, filed on Sep. 24, 2020, now abandoned.
Application No. 17/041,119 is a continuation of application No. PCT/AU2029/050277, filed on Mar. 28, 2019.

(30) Foreign Application Priority Data
Mar. 28, 2018 (AU) 2018901033

(51) Int. Cl. A61M 5/42; A61B 17/132; A61F 13/02; A61H 7/00; A61H 23/02; A61M 35/00; A61N 1/36

(52) U.S. Cl. CPC A61M 5/422; A61F 13/02; A61H 7/001; A61M 35/006; A61N 1/36021; A61B 17/132; A61H 23/0236; A61H 2201/02; A61H 2201/105; A61H 2201/1695; A61M 2202/048

(57) ABSTRACT

A device for reducing the pain associated with an injection is described, including a body that is configured in use to be positioned around or proximal to an injection site while allowing a user to view the injection site while performing the injection, the body including an operative face configured to be placed against the skin of a user and having a stimulus element in the form of at least one protrusion configured to extend beyond the operative face to contact the skin of the user around or proximal to the injection site and stimulate the skin of the user.

### TECHNICAL FIELD

A device for reducing patient or user distress during an injection is disclosed.

### BACKGROUND ART

The pain and distress that result from administering active substances by injection or taking samples using a hypodermic needle or other minor procedures is a significant cause of stress in the health care field. Childhood immunizations tend to result in fear and anxiety in the patient and those around them, and can cause long term fears. Older patients too, can have a fear of injections that may decrease rapport with a health care professional and hinder a health care professional's ability to do their job. It may even result in an individual not attending a medical service when needed. There is significant benefit in procedure and/or device that diminishes the pain or distress associated with injections.

There is particular benefit to individuals who need to take regular injections in order to maintain their levels of insulin, testosterone, or other hormones. The pain elicited by chronic injections can be a constant source of anxiety and stress to the individual. In some cases, such pain can keep the patient from following a more aggressive protocol just so they can avoid the increased number of injections. It will be appreciated that any device and/or procedure that diminishes the pain associated with daily or more than daily injections would be of great help.

There are several techniques used by health care professionals to minimise the pain and distress associated with injections and minor procedures. Some professionals use distraction techniques, such as asking the patient to cough or attempting to draw their visual attention away from the injection site. There are also visual barriers which prevent the person from looking at the injection site. However, these distraction techniques do not reduce or mask the pain of the injection, and there is still a risk of the patient having a bad fear induced reaction.

Current studies have supported the gate control theory of pain awareness and transmission. Pain impulses are transmitted by through the spinal cord to the brain. Gate theory states that there is a gating mechanism in the spinal cord that acts to facilitate or inhibit transmission of pain signals to the brain. These “gates” are effected by many things including stimulation of alternative sensory nerve fibres, such as those that respond to mechanical stimuli of touch and vibration.

Practical application of gate control theory has led to the development and widespread use of transcutaneous electrical nerve stimulation or TENS units which stimulate sensory fibres with repetitive electrical impulses to use gate control to block pain signals. In many individuals with chronic pain TENS units employing the gate control theory of pain management provide significant and predictable relief from their pain. However TENS units are costly and tend not to be available for use with injections.

Anecdotal experience by health care professionals has shown that the local application of pressure can reduce the pain from simple injections. However, this pressure requires a health care professional to have a free hand that can provide pressure.

It would be beneficial to provide a device that allows for stimulation in one or more ways while allowing a health care professional to operate without limitation.

### SUMMARY

According to a first aspect, disclosed is a device for reducing the pain associated with an injection or minor procedure, the device comprising: a body configured in use to be positioned around or proximal to an injection site while allowing a user to view the injection site while performing the injection, the body comprising an operative face configured to be placed against the skin of a user; the body having: a first stimulus element in the form of at least one protrusion configured to extend beyond the operative face to contact the skin of the user around or proximal to the injection site and stimulate the skin of the user.

In some forms the device includes at least one secondary stimulus element.

The device has the advantages of providing stimuli that mask or reduce pain impact on a user and allow a health care professional to position the device and then have hands free for the injection or procedure. The device also allows for a combination of various stimuli such as chemical, thermal, physical, auditory, electrical or visual which can work together to provide sufficient stimulation.

In a second aspect, disclosed is a device for reducing the pain associated with an injection or minor procedure, the device comprising: a body configured in use to be positioned around or proximal to an injection site while allowing a user to view the injection site while performing the injection, the body comprising an operative face configured to be placed against the skin of a user; the body having: a stimulus element in the form of at least one protrusion configured to move between a retracted position and an extended position in which the protrusion extends beyond the operative face to contact the skin of the user around or proximal to the injection site and stimulate the skin of the user. The movement between a first and second position may be through mechanical pressing or through relative expansion or retraction of one part in relation to another.

The device has the advantages of providing a stimulus that masks or reduce pain impact on a user without the danger of stimulating the skin early or breaking skin prior to use.

### BRIEF DESCRIPTION

Notwithstanding any other forms that may fall within the scope of the disclosure, specific embodiments will now be described, by way of example only, with reference to the accompanying drawings in which:

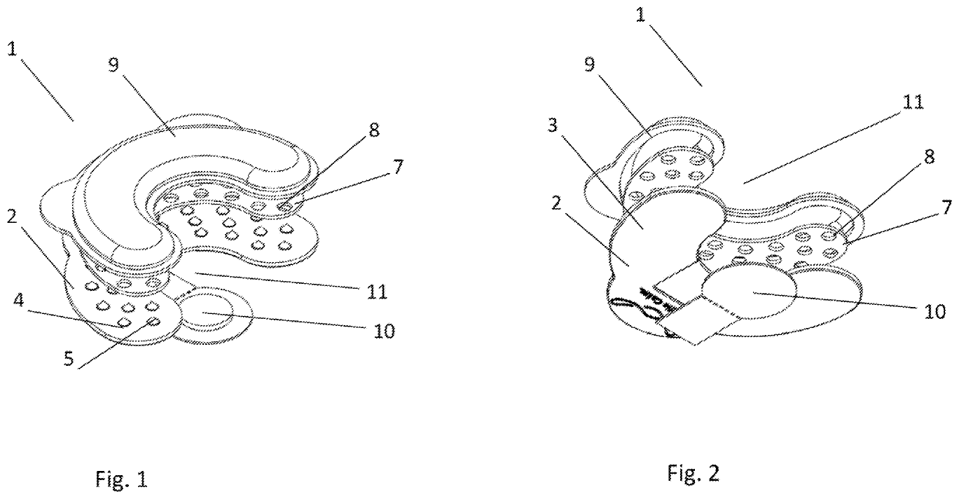

FIG. 1 shows a top perspective view of an embodiment of the pain management device;

FIG. 2 shows a bottom perspective view of the device of FIG. 1;

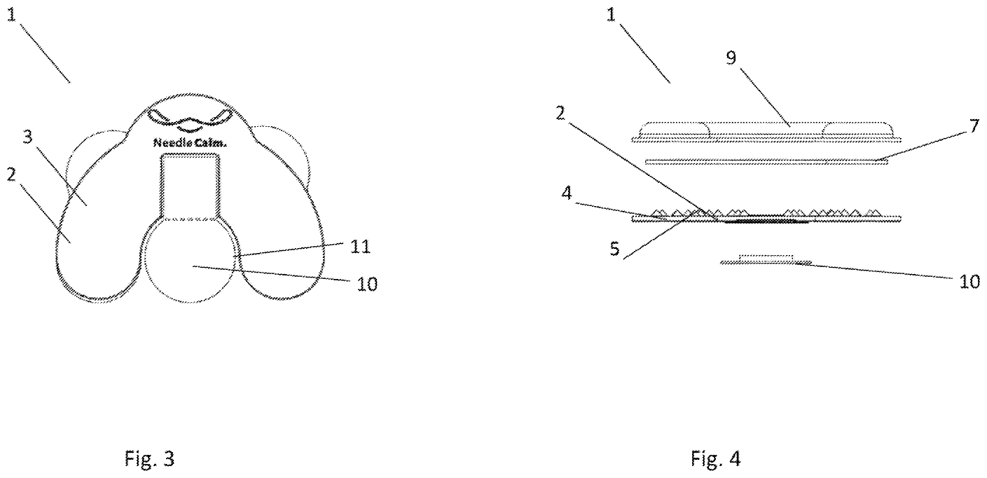

FIG. 3 shows a top view of the device of FIG. 1;

FIG. 4 shows a side view of the device of FIG. 1;

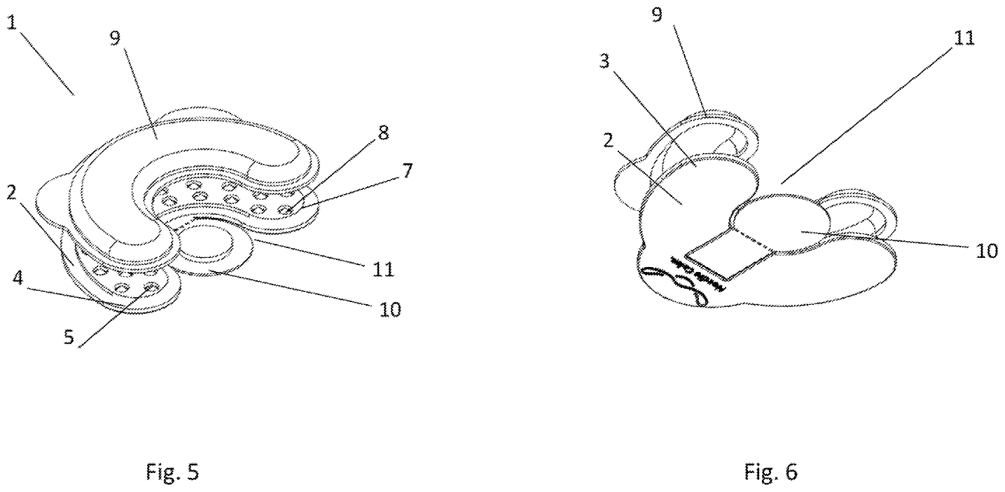

FIG. 5 shows a top perspective view of the device of FIG. 1 partially assembled with cover off;

FIG. 6 shows a bottom perspective of the device of FIG. 5;

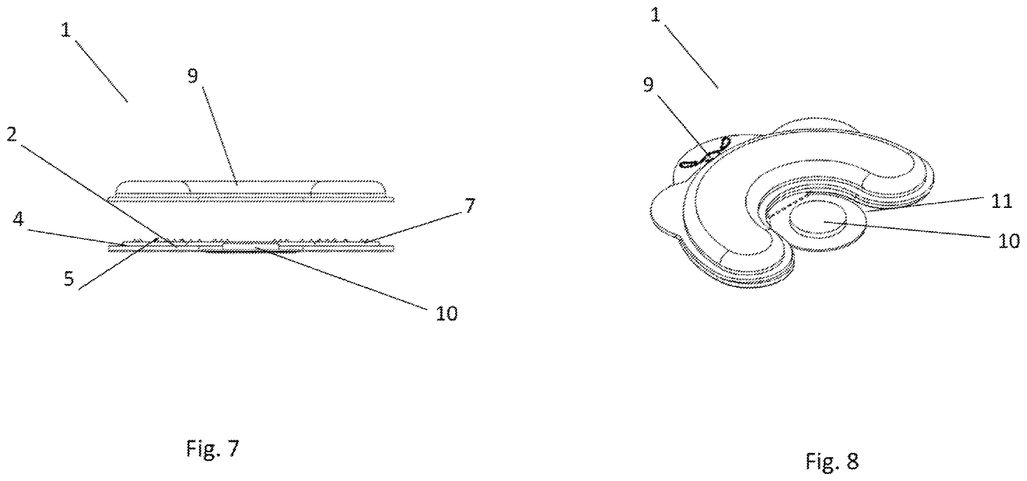

FIG. 7 shows a side view of the device of FIG. 5;

FIG. 8 shows a top perspective view of the device of FIG. 1, assembled and closed;

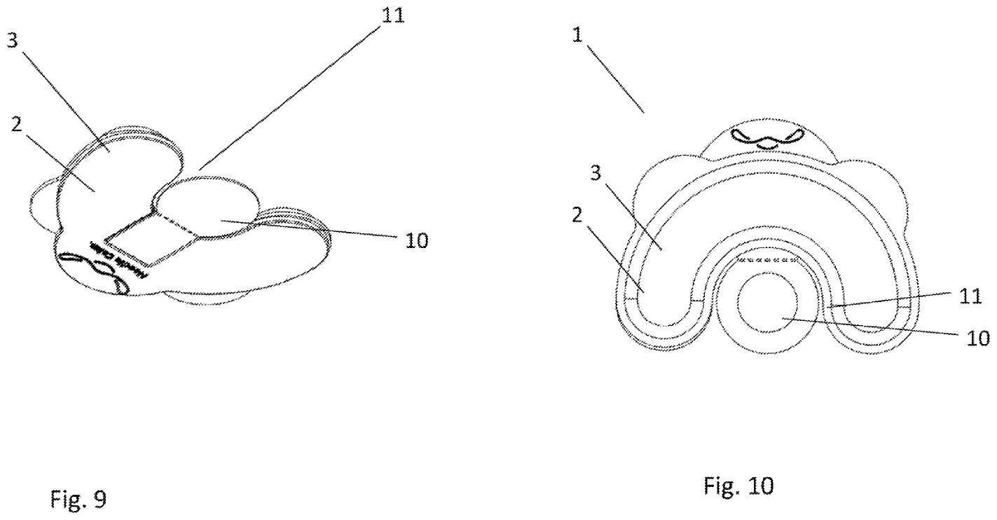

FIG. 9 shows a bottom perspective view of the device shown in FIG. 8;

FIG. 10 shows a side view of the device as shown in FIG. 8;

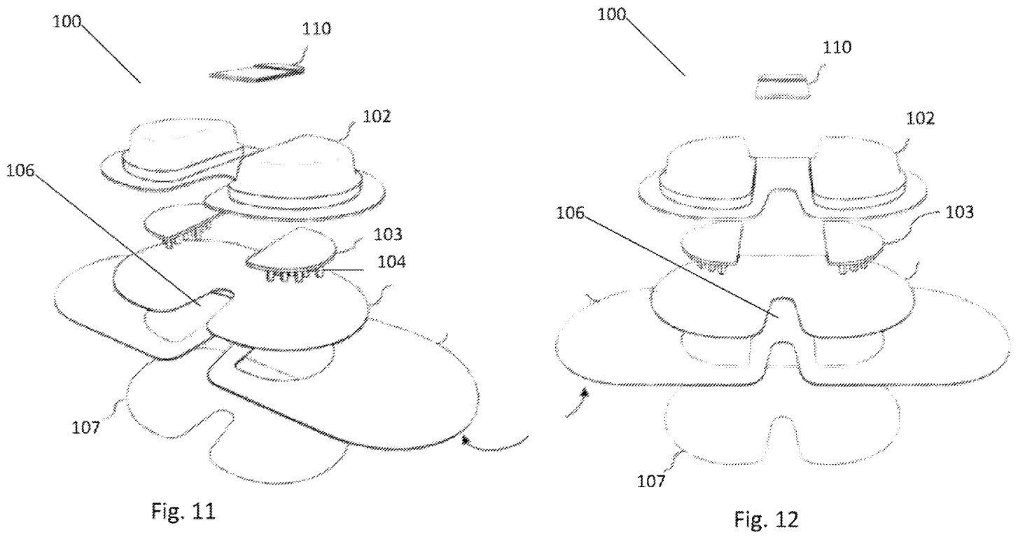

FIG. 11 shows a top perspective view of a second embodiment of the pain management device of the disclosure;

FIG. 12 shows a front perspective view of the embodiment of FIG. 11;

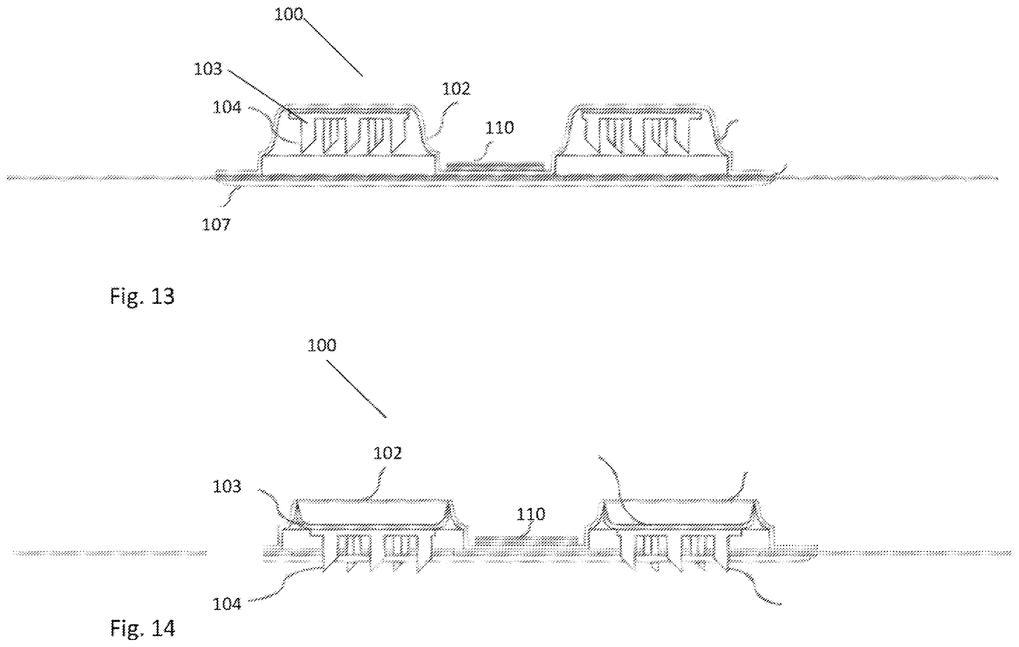

FIG. 13 shows a side cross sectional view of the embodiment of FIG. 11;

FIG. 14 shows a side cross sectional view of the embodiment of FIG. 11 in use;

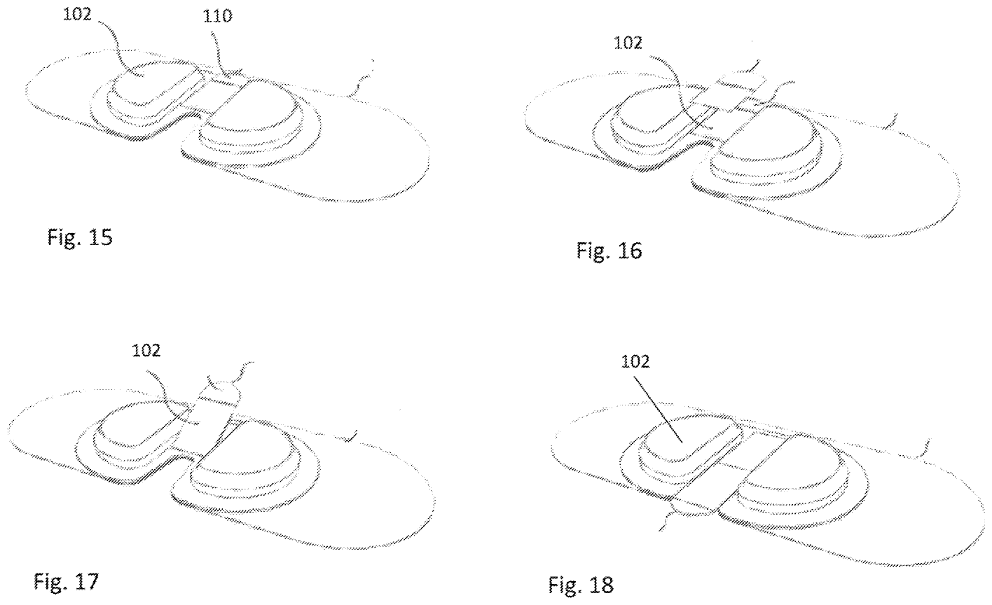

FIG. 15 shows a front perspective view of the embodiment of FIG. 11;

FIG. 16 shows a front perspective view of the embodiment of FIG. 11;

FIG. 17 shows a front perspective view of the embodiment of FIG. 11;

FIG. 18 shows a front perspective view of the embodiment of FIG. 11;

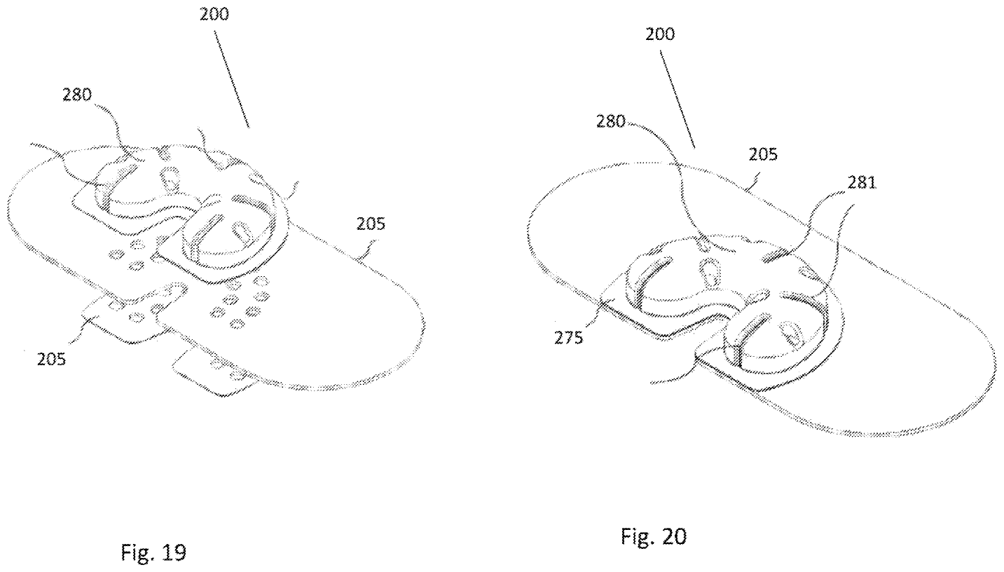

FIG. 19 shows a front perspective view of a third embodiment of the device;

FIG. 20 shows a front perspective view of the embodiment of FIG. 19 with a cover and fully assembled;

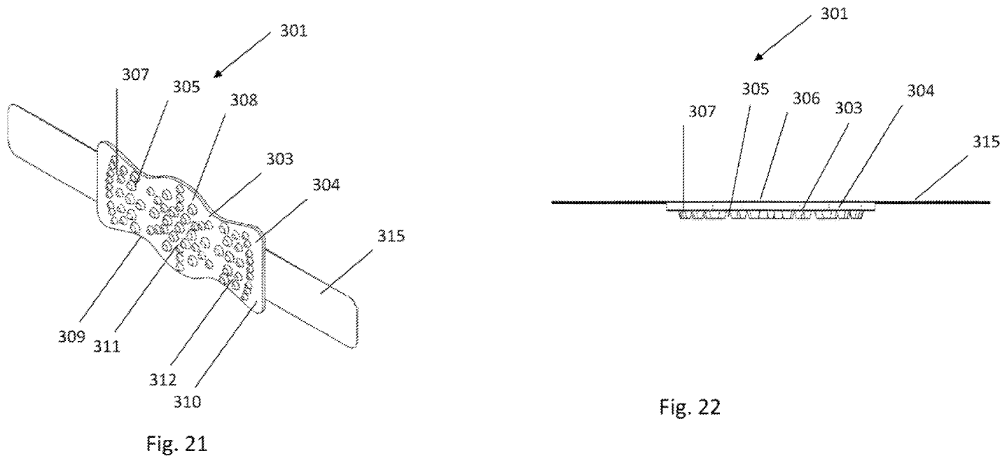

FIG. 21 shows a perspective view of a fourth embodiment of the device;

FIG. 22 shows a side view of the embodiment of FIG. 21;

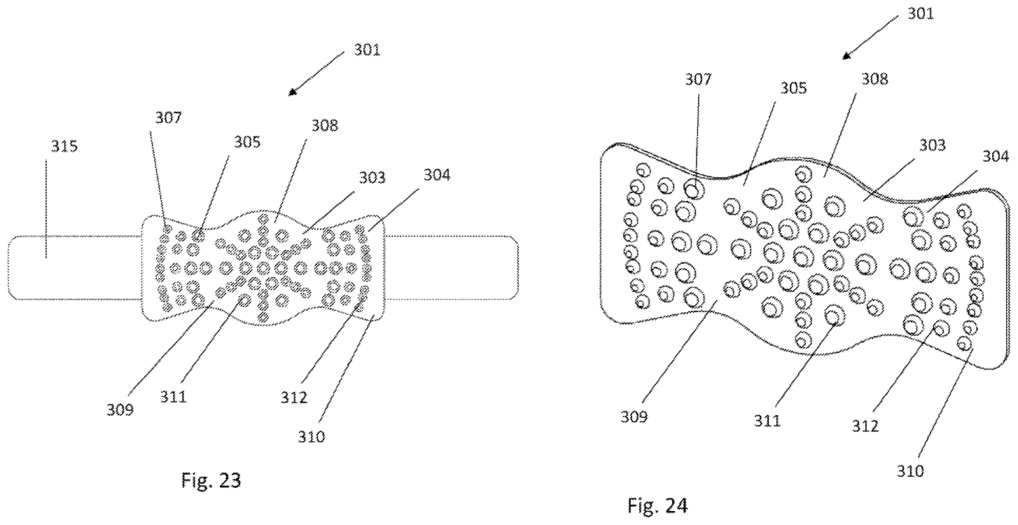

FIG. 23 shows a top view of the embodiment of FIG. 21;

FIG. 24 shows a perspective view of further embodiment of the device;

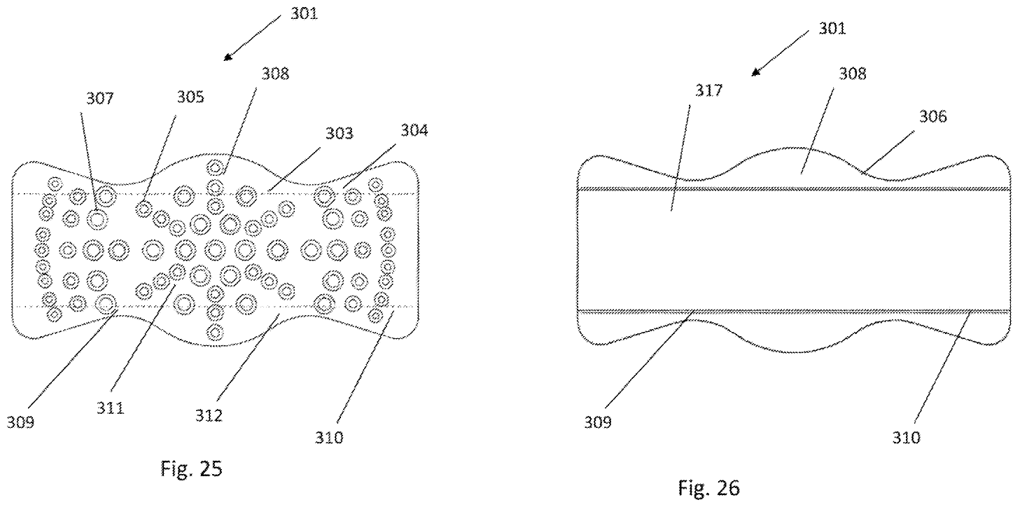

FIG. 25 shows a top view of the device of FIG. 24;

FIG. 26 shows a bottom view of the device of FIG. 24.

### DETAILED DESCRIPTION

Disclosed in some forms is a device for reducing the pain associated with an injection, the device comprising: a body configured in use to be positioned around or proximal to an injection site while allowing a user to view the injection site while performing the injection, the body comprising an operative face configured to be placed against the skin of a user; the body having: a first stimulus element in the form of at least one protrusion configured to extend beyond the operative face to contact the skin of the user around or proximal to the injection site and stimulate the skin of the user; and, at least one secondary stimulus element.

In some forms the body is configured to be secured to the user such that it is retained in positioned around or proximal to the injection site.

In some forms the body is secured to the user by means of an adhesive on at least a portion of the operative face.

In some forms the body is secured to the user by means of a strap.

In some forms the body is shaped to partially surround the injection site.

In some forms the body is substantially C shaped to provide an injection site opening.

In some forms the device as defined in any one of the preceding claims, wherein the at least one protrusion comprises a plurality of protrusions extending from or through the operative face.

In some forms at least a portion of the plurality of protrusions is in the form of spikes or barbs extending from or through the operative face.

In some forms at least a portion of the plurality of protrusions is moveable between a retracted position and an extended position.

In some forms the protrusions are configured to extend through a spacing layer such as a pad or reservoir positioned on the body.

In some forms at least a portion of the protrusions are moveable from a position in which the pad or reservoir covers the protrusions to a position in which the protrusions extend beyond the pad or reservoir.

In some forms the secondary stimulus element is in the form of an active substance.

In some forms the active substance comprises a composition having a stimulating effect on the skin.

In some forms the active substance is a topical analgesic, anaesthetic or a natural or herbal product.

In some forms the active substance is stored in padding located on the operative face of the body.

In some forms the device further comprises a cover adapted to cover the operative face of the body.

In some forms the operative face of the body has padding attached thereto and the cover is shaped to cover the padding.

In some forms the padding retains an active substance in use and the cover is designed to prevent compression of the padding.

In some forms wherein the secondary stimulus element provides a thermal stimulus to the skin proximal the injection site.

In some forms the secondary stimulus element provides a sonic or other vibratory stimulus to the skin proximal the injection site.

In some forms the secondary stimulus element provides electrical stimulation to the nerves proximal the injection site.

In some forms the device is incorporated into a tourniquet.

In some forms an adhesive bandage extends from the body and can be moved into position to cover the injection site.

Further disclosed is a device for reducing the pain associated with an injection, the device comprising: a first stimulus layer, having protrusions extending therefrom; a second spacing layer positioned over the stimulus layer, a removable cover covering the layers and removable for use, the device being configured such that the first stimulus layer and second spacing layer are aligned so that in use the cover is removed, the device is positioned with the padding layer in contact with the skin, and pressure is placed on the stimulus layer such that the protrusions of the stimulus layer extend through the spacing layer to contact the skin of a user.

Disclosed is a device for reducing the pain associated with an injection or minor procedure, the device comprising: a body configured in use to be positioned around or proximal to an injection site while allowing a user to view the injection site while performing the injection, the body comprising an operative face configured to be placed against the skin of a user; the body having: a stimulus element in the form of at least one protrusion configured to move between a retracted position and an extended position in which the protrusion extends beyond the operative face to contact the skin of the user around or proximal to the injection site and stimulate the skin of the user.

In some forms the spacing layer is in the form of padding. In some forms the padding is configured to retain an active substance.

In some forms the material of the device is flexible and mouldable without distortion. In some forms the material can be cooled or heated without effecting the flexibility or mouldable characteristics. In some forms the material is transparent and allows for viewing of the skin through the device to inspect for hematoma, skin irritation or other desirable or undesirable results. In some forms the material is silicon. In some form the device includes a capsule containing active fluids.

Further, disclosed is a device for reducing the pain associated with an injection, the device comprising: a body configured in use to be positioned around or proximal to an injection site while allowing a user to view the injection site while performing the injection, the body comprising an operative face configured to be placed against the skin of a user; the body having: a first stimulus element in the form of an active substance stored in the device and released, the active substance being selected from a group of active substances that will stimulate the skin or nerves on contact and reduce or mask the pain of the injection; at least one secondary stimulus element.

Referring now to FIGS. 1-10, disclosed is a pain management device 1 for reducing, limiting or otherwise masking the pain of an injection or sample using a hypodermic needle. The overall shape of the device 1 is substantially a C-shape having an injection site opening 11 within the C shape. The device is adapted to be positioned proximal to an injection site such that the injection site is positioned and visible within the opening 11.

The device 1 comprises a first supporting layer 2 which is C shaped. The supporting layer 2 comprises a backing face 3 and a proximal or operative face 4. In use the operative face 4 is adapted to be positioned in a facing relationship with the skin of a user. As seen best in FIG. 1, the operative face in this illustrated form includes a plurality of protrusions 5. In this form the protrusions 5 are in the form of multiple spaced apart barbs or spikes extending outwardly from the operative face 4. The barbs or spikes may have any pattern or may be collected in one region. It will be clearly understood that these protrusions may be in the form of single or multiple protrusions having a shape that is designed to stimulate skin when it comes into contact with the skin. In the illustrated form the protrusions 5 are spread across the whole surface of the operative face 4. In this form the protrusions end with a pointed tip, although it will be clear that other shapes are viable for the purpose.

The device 1 further comprises a second spacing layer 7 which is shaped to align with the supporting layer in this form. The spacing layer 7 separates the protrusions from the skin of a user until the device is actuated. In some forms the spacing layer 7 is in the form of padding or alternative material.

In the illustrated form the spacing layer 7 includes a plurality of orifices 8 which are positioned to align with the protrusions 5 of the operative face 4. In some forms these orifices are not required and the protrusions 5 break through the spacing layer or push against the spacing layer such that both layers move.

In some forms, the spacing layer is in the form of a reservoir or padding for retaining an active substance. The active substance may have a stimulating effect on the skin or nerves of the patient. For example the active substance could be a herbal having stimulating properties, cooling properties or other properties. Alternatively the active substance could be a topical analgesic or anaesthetic.

The device 1 further includes a cover 9 which is shaped to cover the protrusions and spacing layer and to prevent pressure on the device. In some forms the cover is curved to cover and not press against the protrusions and spacing layer. In some forms the cover is rigid to protect the protrusions and spacing layer before use.

In use as best shown in FIGS. 5-10, the cover 9 is removed from the device. The device is then positioned in contact with the skin around or proximal the injection site such that the backing face 3 of the supporting layer 2 faces away from the skin and the operative face 4 of the supporting layer 2 faces toward the skin.

Pressure on the backing face 3 of the device results in the protrusions 5 of the operative face moving into contact with the skin through the spacing layer 7. The protrusions 5 place pressure on the skin and stimulate the skin to reduce a pain reaction to the injection or needle. The pressure will also, in some forms, expel the active substance from the spacing layer 7.

The device 1 further includes an adhesive bandage 10 which is located over the opening 11 such that it can be folded back for the injection, then folded back over the injection site at opening 11 to be sealed to the patient's skin. In some forms the adhesive bandage is a small bandage shaped to fit within the opening 11 and includes a frangible tear line 12 for easy removal from the device.

A second embodiment of the pain management device 100 is shown in FIGS. 11-18. In this form, the device 100 has two substantially hemispherical shaped wings 102 forming a main body and rear cover. A stimulus element 103 is located in each wing 102 and includes a plurality of projections 104. The pain management device 100 comprises several layers of different materials. An opening 106 is located between the wings in this form to allow an injection or procedure site to be positioned between the wings and to be viewed by the medical professional.

In this form, an active substance is retained in sealed reservoirs formed in the main body and rear cover 102. The stimulus element 103 therefore has two roles, that of stimulating the patient's skin in use and breaking a lower seal 105 of the reservoir within the cover 102.

Attached to the underside of the device 100 is a padding layer 107. The padding layer 107 is made of porous material such as sponge or foam or other suitable material that is able to absorb a flowable active substance held in the reservoirs such as an anaesthetic or analgesic substance. An adhesive bandage 110 may also be included.

Referring to FIGS. 13 and 14, when the pain management device 100 is in use the user pushes each of the two chambers formed by the backing cover 102 down. The user collapses the chambers and causes the projections 104 of the stimulus element 103 to puncture the lower sealing member 105 as seen in FIG. 14. The stimulus elements 103 project downwardly extend through the padding layer 107 so as to come in contact with the skin of the patient. The reactive substance is released through the holes punctured in the lower sealing member 105 thus providing fluid communication between the reservoirs holding the active substance and the padding layer 107. As the reactive substance is released into the padding layer 107 through the holes it spreads quickly in the porous padding layer and reaches the patient's skin. The active substance spreads around and through the padding layer 107 and numbs or stimulates or otherwise impacts the patient's skin under it. This structure of the pressure members puncturing the lower sealing member allows for the active substance to be administered while the pain management device is in use. The user could choose to puncture the lower sealing member first but the projections of the pressure members are intended to be of appropriate height so that they will not inflict pain to the patient when the lower sealing member is punctured even when the pain management device is attached to the patient's skin. The height of the pressure members will be approximately 20 mm but can vary depending on the size of the reservoir and thickness of the padding layer.

The pain management device 100 may further have a protective film 109 on top of the backing cover 102. This protective layer is made of resilient material such as silicone or rubber that allows pressing the upper sealing layer 102 towards the lower sealing layer 105 and the protective film returns to its original shape once pressing stops.

As seen in FIGS. 15-18, the pain management device 100 further includes an adhesive bandage 110. The bandage 110 is in the form of a flap attached to the end of the opening 104 of the device 100. Its other end is releasably attached to a portion of the device. FIG. 16 shows a peelable plastic or paper liner 112 that exposes an adhesive section 113 and a padded section 114 of the flap when removed. Once the procedure or injection has been finished, the flap is closed over the opening 104 so as to cover the site of the procedure. The padding layer 114 on the flap 113 covers the procedure site, thus removing the need for a cotton ball or a plaster. Having the flap readily attached to the pain management device reduces the total time needed for a procedure and it minimises the need for other products such as dressings or plasters.

In each of the exemplary embodiments of the present invention, the rate at which the active substance is released from the device can be configured such that it will provide the desired benefits for at least the entire procedure. Also the time between releasing an active substance, pressing the protrusions against the skin and performing the injection or procedure can be varied as needed.

The active substance used can be any topical anaesthetic, analgesic, natural or herbal product known in the art, or a combination thereof. For example, vanillyl butyl ether, benzocaine, lidocaine, or other amino esters or amino amides suitable for topically administered pain relief can be used. An advantage of topical anaesthetics is that they are effective very quickly so the procedure can take place within minutes after placing the pain management device on the patient's skin. The strength and amount of anaesthetic depends on the size of the reservoirs and the thickness and absorption ability of the padding layer. The amount of anaesthetic should be sufficient to numb the patient's skin temporarily allowing more comfort to the patient whilst considering that any excess would overflow from under the pain management device. In addition, other types of suitable substances such as natural products may be used to provide a degree of comfort to the user. An example of a suitable natural product is liniments such as Tiger Balm™ or Deep Heat™, or Kwan Loong™ medicated oil. Liniments are known to work similarly to pressure applied on the skin. Therefore, applying a liniment overstimulates the nerves causing the nerve receptors to shut down, thus blocking the pain signal from being transmitted to the brain.

A third embodiment of the pain management device 200 is seen in FIGS. 19 and 20. In this embodiment, the pain management device 200 has one peanut-shaped reservoir for containing the active substance and the pressure member. The peanut-shaped reservoir has an upper sealing member 280 that forms an upwardly protruding chamber, and a lower sealing member 275. The lower sealing member 275 is a sheet of material attached to the underside of the upper sealing member 280 at its peripheral regions surrounding the peanut-shaped chamber. The lower sealing member 275 is attached to the main body 205 so that the peanut-shaped reservoir surrounds the cut-out section of the main body. The main body 205 has multiple apertures located around the cut-out section and under the lower sealing member 275. A padding layer 235 is attached to the underside of the main body 205 and this padding layer has multiple apertures that coincide with the apertures of the main body 205. The padding layer 235 also has a cut-out section coinciding with the cut-out section of the main body 205 so that the cut-out section forms a horse shoe-shape around the procedure site when the pain management device is in use.

As seen in FIG. 20, a pressure member is contained within the reservoir filled with the active substance. The pressure member is preferably peanut-shaped and fits snugly within the reservoir formed by the upper and lower sealing members 280, 275. In this embodiment, the pressure member has multiple tubular projections that each have a slanted end so as to make the downwardly protruding projections pointed. The projections of the pressure member 260 coincide with the holes in the main body 205 and the padding layer 235 so that when the pressure member is pressed down the projections pierce through the lower sealing member and then through the holes of the main body and the padding layer. Once the pressure member has punctured the lower sealing member the active substance is free to flow through the holes of the main body into the padding layer adjacent the patient's skin.

As seen in FIGS. 19 and 20, the upper sealing member 280 includes several indentations 281 that provide weakened areas for the upper sealing member 280. These indentations allow the chamber formed by the upper sealing member to be pushed down more easily so that the user can effortlessly push the pressure member 260 down through the lower sealing member 275.

Referring now to FIGS. 21 through 23, disclosed is pain management device 301 for reducing, limiting or otherwise masking the pain of an injection or sample using a hypodermic needle. The device is adapted to be positioned proximal to an injection site, such as between the injection site and a tourniquet or between the injection site and the brain. The device is configured such that the injection site is clearly visible.

The device 301 comprises a contact member 303. The contact member comprises a support 304 having an operative face 305. The support also has a backing face 306. In use the operative face 305 is adapted to be positioned in a facing relationship with the skin of a user. The contact member 303 is in the form of an elongate tab having a profiled edge in the shape of a wave. The tab therefore has a central expanded region 308, tapered waists 309 either side of the central expanded region, and flared ends 310.

The operative face in this illustrated form includes a plurality of protrusions 307. In this form the protrusions 307 are in the form of multiple spaced apart pillars extending outwardly from the operative face 305. The protrusions are essentially transverse to the plane of the operative face. The pillars may have any pattern or may be collected in one region. The pillars may have blunt ends as shown in the illustrated form. Alternatively, the pillars may have sharp or pointed ends or a variety of ends. The pillars may have a variety of sizes and shapes.

As shown in the illustrated form, the pillars are in the form of a wagon wheel 311 of pillars extending outwardly from one point in the central expanded section, with further C shaped formations 312 of pillars towards the flared ends 310. It will be clearly understood that these protrusions may be in the form of single or multiple protrusions having a shape that is designed to stimulate skin when it comes into contact with the skin.

The device 301 in some forms may include an adhesive on at least a portion of the operative face 305. A chemical stimulant or anaesthetic such as, for example any topical anaesthetic, analgesic, natural or herbal product known in the art, or a combination thereof may be deposited on the operative face. For example, vanillyl butyl ether, benzocaine, lidocaine, or other amino esters or amino amides suitable for topically administered pain relief can be used. The amount of anaesthetic should be sufficient to numb the patient's skin temporarily allowing more comfort to the patient whilst considering that any excess would overflow from under the pain management device. In addition, other types of suitable substances such as natural products may be used to provide a degree of comfort to the user. An example of a suitable natural product is liniments such as Tiger Balm™ or Deep Heat™, or Kwan Loong™ medicated oil. Liniments are known to work similarly to pressure applied on the skin. Therefore, applying a liniment overstimulates the nerves causing the nerve receptors to shut down, thus blocking the pain signal from being transmitted to the brain.

The device further includes a strap or tape 315 which is removably engaged with the contact member 303. The strap or tape may be in the form of micropore tape or other known tape. As best shown in FIG. 26, the backing face 306 includes a tape positioning recess 317. The device may be used with or without the strap or tape.

In the illustrated form the spacing layer 7 includes a plurality of orifices 8 which are positioned to align with the protrusions 5 of the operative face 4. In some forms these orifices are not required and the protrusions 5 break through the spacing layer or push against the spacing layer such that both layers move.

In some forms, the spacing layer is in the form of a reservoir or padding for retaining an active substance. The active substance may have a stimulating effect on the skin or nerves of the patient. For example, the active substance could be a herbal having stimulating properties, cooling properties or other properties. Alternatively the active substance could be a topical analgesic or anaesthetic.

The device 1 further includes a cover 9 which is shaped to cover the protrusions and spacing layer and to prevent pressure on the device. In some forms the cover is curved to cover and not press against the protrusions and spacing layer. In some forms the cover is rigid to protect the protrusions and spacing layer before use.

In use as best shown in FIGS. 5-10, the cover 9 is removed from the device. The device is then positioned in contact with the skin around or proximal the injection site such that the backing face 3 of the supporting layer 2 faces away from the skin and the operative face 4 of the supporting layer 2 faces toward the skin.

Pressure on the backing face 3 of the device results in the protrusions 5 of the operative face moving into contact with the skin through the spacing layer 7. The protrusions 5 place pressure on the skin and stimulate the skin to reduce a pain reaction to the injection or needle. The pressure will also, in some forms, expel the active substance from the spacing layer 7.

The device 1 further includes an adhesive bandage 10 which is located over the opening 11 such that it can be folded back for the injection, then folded back over the injection site at opening 11 to be sealed to the patient's skin. In some forms the adhesive bandage is a small bandage shaped to fit within the opening 11 and includes a frangible tear line 12 for easy removal from the device.

Advantageously, the pain management device is a sterile, single use product, and as such there is no clean up required, which minimises infection control issues.

Advantageously, the upper surface of the pain management device may be provided with images or other art work intended to appeal to children.

In some forms, the device may be configured to be secured to the patient by means of a tourniquet or sleeve to which the device is attached or into which the device is built.

In some forms the device may include alternative stimulus. In some forms that stimulus may be in the form of sonic vibration or electrical stimulus. Alternatively thermal stimulus such as cooling may be utilised. For example the material may be able to be cooled or frozen without changing the mouldable nature of the device. The temperature of the device may be indicated through colour change materials. In other forms a visual stimulation such as lighting or strobe effects to act as distraction or colour change or visual appeal may be utilised to block pain gateways or to otherwise mask or reduce pain.

In some forms the temperature or pressure on the spikes or pillars may have an impact on the hardness of the pillar. The deformity of the protrusions may relax with time or temperature to allow for a shifting pressure that maintains stimulation of the skin. In some forms protrusions could extend or retract with heat or cold or other changes resulting in an ongoing movement and stimulation. In one embodiment lateral movement of the protrusions could result from temperature or other changes resulting in changing stimulation.

In some forms two separate materials could be utilised for the protrusions and a protective layer such that when the device is heated, cooled or otherwise changed the protrusions may expand more quickly than the protective layer allowing for stimulation. In some forms an edge of the device has an impact on the stimulation rather than just the protrusions.

In some forms the electrical, thermal or visual stimulation may be driven by a smart device or a dedicated device. An app may be utilised to control the provision of stimulus and may be associated with a picture, a video or an audio image.

In some forms the system may provide biofeedback such as measuring pulse or sweat.

In some forms the form of the various stimuli elements may be selectable by the user.

It will be understood to persons skilled in the art that many other modifications may be made without departing from the spirit and scope of the disclosure herein.

In the claims which follow and in the preceding description, except where the context requires otherwise due to express language or necessary implication, the word “comprise” or variations thereof such as “comprises” or “comprising” is used in an inclusive sense, i.e. to specify the presence of the stated features but not to preclude the presence or addition of further features in various embodiments of the actuator, method of manufacturing an actuator and composition as disclosed herein.

1. A device for reducing the pain associated with an injection, the device comprising:
a body in the form of an elongate tab having an operative face and a backing face, the operative face configured in use to be positioned on a patient's skin, allowing a user to select an injection site near the body and view the injection site while performing the injection;
the body having:
a stimulus element in the form of a plurality of protrusions configured to extend beyond the operative face to contact the skin of the patient around or proximal to the injection site and stimulate the skin of the patient;
the elongate tab having two profiled edges such that the tab comprises a central expanded region, tapered waists either side of the central expanded region and flared ends.

2. The device as defined in claim 1, further comprising a second stimulus element.

3. The device as defined in claim 1, wherein the body is configured to be secured to the patient such that it is retained in position proximal to the injection site.

4. The device as defined in claim 1, wherein at least a portion of the plurality of protrusions is in the form of bumps, spikes or barbs extending from or through the operative face.

5. The device as defined in claim 4, wherein the secondary stimulus element is in the form of an active substance.

6. The device as defined in claim 5, wherein the active substance comprises a composition having a stimulating effect on the skin.

7. The device as defined in claim 5, wherein the active substance is a topical analgesic, anaesthetic or a natural or herbal product.

8. The device as defined in claim 5, wherein the active substance is stored in padding located on the operative face of the body.

9. The device as defined in claim 1, further comprising a cover adapted to cover the operative face of the body.

10. The device as defined in claim 9, wherein the operative face of the body has padding attached thereto and the cover is shaped to cover the padding.

11. The device as defined in claim 9, wherein the padding retains an active substance in use and the cover is designed to prevent compression of the padding.

12. The device as defined in claim 1, wherein an adhesive bandage extends from the body to cover the injection site.

13. The device as defined in claim 1, wherein in use the injection site is located external to an outer edge of the body of the device and adjacent the body.
